(19) United States
(12) Patent Application Publication
(10) Pub. No.: US 2007/0275902 A1
(43) Pub. Date: Nov. 29, 2007

(54) Treatment of Phosphatidylinositol Phospholipid Disorders

(75) Inventors: Matthew A. Gonda, Birmingham, AL (US); Vernon Leon Alvarez, Morrisville, PA (US); Carol A. Grimes, Raleigh, NC (US)

Correspondence Address: CHOATE, HALL & STEWART LLP, TWO INTERNATIONAL PLACE, BOSTON, MA, 02110, US

(73) Assignee: TRANSMOLECULAR, INC., CAMBRIDGE MASSACHUSETTS, MA (US)

(21) Appl. No.: 10/580,302

(22) PCT Filed: Nov. 23, 2004
(86) PCT No.: PCT/US04/39325

§ 371 Date: May 22, 2007

Related U.S. Application Data
(60) Provisional application No. 60/524,884, filed on Nov. 26, 2003.

(51) Int. Cl. A61K 38/08; A61K 38/02; A61P 35/00; A61P 35/02; C12N 5/06; C12N 5/08; G01N 33/574

(52) U.S. Cl. 514/16; 435/375; 435/7.23; 514/2

(57) ABSTRACT

Disclosed is a method of treating cell proliferative disorders, such as cancer, with amounts of chlorotoxin and/or derivatives, analogs or fragments thereof, which are effective to bind to phosphoinositol phospholipids.

### RELATED APPLICATIONS

This application is a national stage application of PCT/US2004/039325, filed Nov. 23, 2004 under 35 USC §371, which claims the benefit of U.S. Provisional Application Ser. No. 60/524,884, filed Nov. 26, 2003, each of which is herein incorporated by reference in their entirety for all purposes. This application is also related to, but does not claim priority to, International Applications PCT/US03/17410 (filed Jun. 2, 2003) and PCT/US03/17411 (filed Jun. 2, 2003) both of which are herein incorporated by reference in their entirety for all purposes.

### FIELD OF THE INVENTION

The present invention relates to a novel method of treating cell proliferative disorders associated with the expression of phosphatidylinositol phospholipids (i.e., phosphatidylinositols) which bind chlorotoxin.

### BACKGROUND OF THE INVENTION

Perhaps the most dominant theme that has emerged in the past decade of signal transduction research is that proteins can become acutely targeted to new cellular locations (usually membranes) by phosphorylation reactions that create binding sites for modular protein domains. The classic example is the Src homology 2 (SH2) domain, which escorts signaling proteins to tyrosine-phosphorylated growth factor receptors or adaptor proteins. In an analogous fashion, phosphorylation of the lipid phosphatidylinositol can create sites for recruitment of proteins to cell membranes. The phosphorylation or hydrolysis of inositol-containing lipids in cell membranes is now known to orchestrate numerous complex cellular events (Corvera et al. (1998) Trends Cell Biol. 8, 442-446; Rameh et al. (1999) J. Biol. Chem. 274, 8347-8350). A variety of domains that recognize specific phosphatidylinositols (phosphorylated forms of phosphatidylinositol) have been described and include pleckstrin homology (PH) domains, FYVE domains, and subsets of gelsolin homology domains, SH2 domains, and PTB domains. Some of these domains exist as large families, and specificity in recruitment is achieved by the ability of individual members of the family to recognize the phosphorylated moiety within distinct structural contexts.

The unique structure of the myo-inositol head group of inositol-containing lipids, with six similar but nonequivalent hydroxy groups, provides a template for formation of multiple phosphorylated species. It is the ability of phosphatidylinositol to form so many distinct phosphorylation and hydrolysis products that has allowed it to evolve into a central regulator in eukaryotic cells. Thus, phosphorylation of phosphatidylinositol at distinct positions on the inositol ring by different kinases results in the production of unique lipids that modulate discrete cellular responses at cell membranes.

The discovery of phosphoinositide 3-kinase (P13K) as an oncoprotein-associated enzyme approximately a decade ago revealed the existence of three additional phosphatidylinositols, phosphatidylinositol-3 -phosphate, phosphatidylinositol-3,4-bisphosphate and phosphatidylinositol-3,4,5-trisphosphate. Previously, only two phosphorylated forms of phosphatidylinositol had been identified, phosphatidylinositol-4-phosphate and phosphatidylinositol-4,5-bisphosphate. In mammalian cells, phosphatidylinositol-3-phosphate is typically five percent as abundant as phosphatidylinositol-4-phosphate or phosphatidylinositol-4,5-P2. Phosphatidylinositol-3,4-bisphosphate and phosphatidylinositol-3,4,5-trisphosphate are nominally absent in quiescent cells and are less than three percent as abundant as phosphatidylinositol-4,5-bisphosphate at peak stimulation. More recently, two additional phosphatidylinositols were found in eukaryotic cells, phosphatidylinositol-5-phosphate and phosphatidylinositol-3,5-bisphosphate (Dove et al. (1997) Nature 390,187-192; Rameh et al. (1997) J. Biol. Chem. 272, 22059-22066; Whiteford et al. (1997) Biochem. J. 323, 597-601). The recent identification of proteins that specifically bind to lipid products of P13K provides insight into the mechanisms by which P13K activation leads to cell growth, survival, transformation, actin rearrangement, vesicle trafficking, and transcriptional regulation (Corvera et al. (1998) Trends Cell Biol. 8, 442-446; Rameh et al. (1999) J. Biol. Chem. 274, 8347-8350).

Chlorotoxin is a thirty-six amino acid protein naturally derived from Leiurus quinquestriatus scorpion venom (DeBin et al. (1993) Am. J. Physiol. 264: C361-369). Compositions (see U.S. Pat. Nos. 5,905,027 and 6,429,187 each of which is hereby incorporated by reference in their entirety) and methods (see U.S. Pat. Nos. 6,028,174 and 6,319,891 each of which is hereby incorporated by reference in their entirety) for diagnosing and treating neuroectodermal tumors (e.g., gliomas and meningiomas) have been developed based on the ability of chlorotoxin to bind to tumor cells including those of neuroectodermal origin (Soroceanu et al. (1998) Cancer Res. 58,4871-4879; Ullrich et al. (1996) Neuroreport 7, 1020-1024; Ullrich et al. (1996) Am. J. Physiol. 270, C1511 -C1521). Diagnosis of tumors is accomplished by identification of labeled chlorotoxin bound to tumor cells while treatment of tumors is accomplished by targeting tumors with chlorotoxin or cytotoxic agents linked to chlorotoxin (see U.S. Pat. No. 6,429,187). The present invention is based upon the identification of a phosphatidylinositol phospholipid receptor expressed on tumor cell membranes which binds chlorotoxin and distinct domains in chlorotoxin which bind to this receptor.

### SUMMARY OF THE INVENTION

The invention includes a method of inhibiting a phosphatidylinositol signaling cascade in a cell comprising contacting a cell expressing a phosphatidylinositol phospholipid with an effective amount of an agent comprising a chlorotoxin binding domain, wherein the chlorotoxin binding domain binds to the phosphatidylinositol phospholipid. In some embodiments the method further comprises contacting the agent with a phosphatidylinositol phospholipid in vitro to determine the effective amount of the agent. In some embodiments, the inhibition of the phosphatidylinositol signaling cascade is effective for the treatment of cancer.

The invention includes a method of inhibiting a phosphatidylinositol signaling cascade in a cell comprising contacting a cell expressing a phosphatidylinositol phospholipid with an agent comprising a chlorotoxin binding domain, wherein the agent is not full length chlorotoxin. In some embodiments, the agent inhibits one or more plasma membrane functions that require the phosphatidylinositol phospholipid. In some embodiments the plasma membrane function is selected from the group consisting of a membrane trafficking function, a membrane-cytoskeletal function and a cell signaling function. In some embodiments the membrane trafficking function is selected from the group consisting of endocytosis and exocytosis while the membrane-cytoskeletal function is selected from the group consisting of microvilli formation and phagocytosis and the cell signaling function is selected from the group consisting of protein kinase activity, GTPase activity and EGFR-dependent membrane ruffling.

The invention includes a method of inhibiting the activity of a cancer cell expressing a phosphatidylinositol phospholipid on the cell surface comprising contacting the cell with an agent comprising a chlorotoxin binding domain. In some embodiments the activity is cancer cell division and cell division is arrested at the G1/S phase of the cell cycle. In some embodiments, the activity is the phosphotidylinositol cell signaling pathway.

The invention includes a method of treating cancer in a patient in need of such treatment comprising administering an effective amount of a composition comprising an agent containing a chlorotoxin binding domain that binds to a cancer cell expressing a phosphatidylinositol phospholipid. In some embodiments, the invention further comprises contacting the agent with a phosphatidylinositol phospholipid in vitro to determine the effective amount of the agent.

A method of treating cancer in a patient in need of such treatment comprising administering an effective amount of a composition comprising an agent containing a chlorotoxin binding domain that binds to a cancer cell expressing a phosphatidylinositol phospholipid, wherein the agent is not full length chlorotoxin.

In some embodiments of the above claimed treatment methods of the invention, the phosphotidylinositol phospholipid is a monophosphate. In some embodiments, the phosphotidylinositol monophosphate is phosphatidyinositol 3-phosphate or phosphatidyinositol 4-phosphate. In some embodiments of the above claimed methods of the invention, the phosphotidylinositol phospholipid is a bisphosphate. In some embodiments, the bisphosphate is phosphatidylinositol 3,4-bisphosphate or phosphatidylinositol 4,5-bisphosphate. In some embodiments of the above claimed methods of the invention, the phosphotidylinositol phospholipid is a trisphosphate. In some embodiments, the trisphosphate is phosphatidylinositol 3,4,5-trisphosphate.

In some embodiments of the above claimed treatment methods of the invention, agent is a polypeptide while in other embodiment, the agent is a peptide mimetic. In some embodiments, the polypeptide comprises at least two chlorotoxin binding domain capable of binding to a phosphotidylinositol phospholipid and the sequence may be KGRGKSY (SEQ ID NO: 8).

In some embodiments of the above claimed methods of the invention, the agent is effective for the treatment of cancer and the cancer is selected from the group consisting of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma.

The invention also includes a method of identifying an agent which binds to phosphatidylinositol phospholipid comprising contacting the phosphatidylinositol phospholipid with the agent in the presence of a polypeptide containing a chlorotoxin binding domain, and detecting the binding of the agent to the phosphatidylinositol phospholipid. In some embodiments, the method further comprising measuring the level of binding of the agent to the phosphatidylinositol phospholipid and/or comparison to a positive and/or negative control.

In some embodiments of the above screening method, the control is a negative control that does not contain a phosphatidylinositol phospholipid while in others the control is a positive control which comprises a phosphatidylinositol phospholipid contacted with chlorotoxin. In some embodiments the chlorotoxin is labeled. In some embodiments, the phosphatidylinositol phospholipid is expressed on the surface of cells and the cells may be exposed to the agent in vitro. These cells may be eukaryotic or prokaryotic cells. In some embodiments, the above method further comprises measuring the level of the phosphatidylinositol phospholipid on the surface of the cells and/or differentiation or proliferation of the cells. In some embodiments, the cells are cancer cells. The cells may be disrupted prior to contact with the agent and the agent may selected from the group consisting of chemical compounds, oligonucleotides, peptides and antibodies and also may be labeled.

In some embodiments of the above claimed screening method of the invention, the phosphotidylinositol phospholipid is a monophosphate. In some embodiments, the phosphotidylinositol monophosphate is phosphatidyinositol 3-phosphate or phosphatidyinositol 4-phosphate. In some embodiments of the above claimed methods of the invention, the phosphotidylinositol phospholipid is a bisphosphate. In some embodiments, the bisphosphate is phosphatidylinositol 3,4-bisphosphate or phosphatidylinositol 4,5-bisphosphate. In some embodiments of the above claimed methods of the invention, the phosphotidylinositol phospholipid is a trisphosphate. In some embodiments, the trisphosphate is phosphatidylinositol 3,4,5-trisphosphate.

### BRIEF DESCRIPTION OF THE DRAWINGS

FIG. 1 depicts structures of phosphatidylinositols and enzymes involved in the phosphatidylinositol pathway.

FIG. 2 depicts a graph summarizing the results from binding experiments demonstrating the binding of chlorotoxin to different phosphatidylinositols.

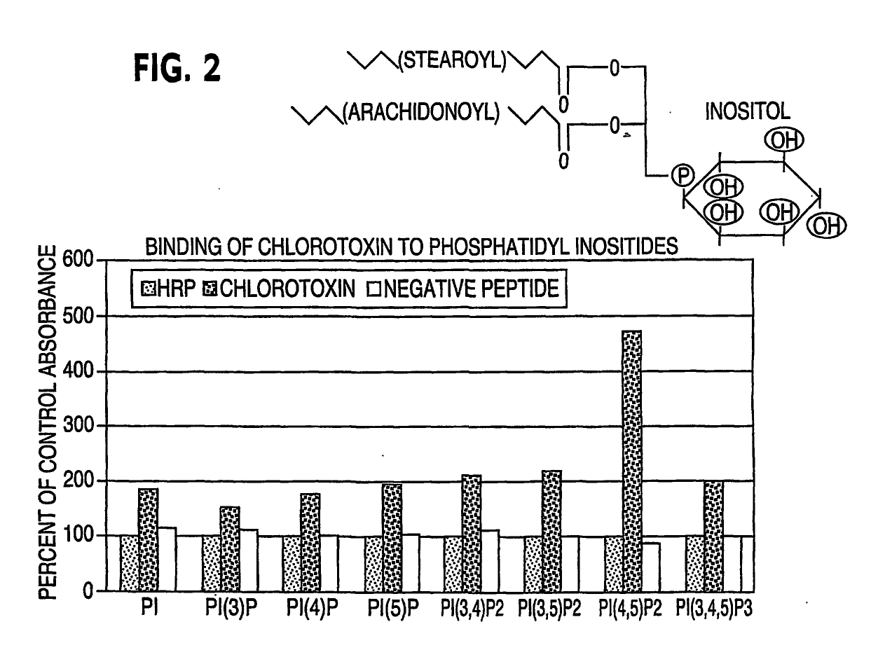

FIG. 3 depicts a graph summarizing the results from binding experiments demonstrating the binding of the beta domain of chlorotoxin (SEQ ID NO: 08) to different phosphatidylinositols.

FIG. 4 depicts a graph summarizing the results from binding experiments demonstrating the absence of binding of the alpha domain of chlorotoxin (SEQ ID NO: 9) to different phosphatidylinositols.

### DETAILED DESCRIPTION OF THE INVENTION

The invention is based in part, on the discovery that chlorotoxin specifically binds to distinct species of phosphatidylinositols. The invention is also based in part, on the identification of binding domains in the chlorotoxin molecule which preferentially interact with distinct species of phosphatidylinositols. The identification of these binding domains has led to the development of screening assays for molecules which display the same properties as chlorotoxin and derivatives of chlorotoxin which display enhanced binding and activity. The binding of chlorotoxin and derivatives thereof to phosphotidylinositols has also led to the discovery that these molecules can be used to modulate the phosphatidylinositol cell signal transduction pathway and treatment of diseases associated with alterations in this pathway.

Methods of Treatment

This invention includes methods for the treatment of a disease characterized by altered phosphatidylinositol expression and/or phosphatidylinositol cell signaling in a cell, comprising administering an amount of an agent containing a chlorotoxin binding domain that is effective to bind or are specific to phosphatidylinositols expressed by these cells. In some embodiments, these cells with altered phosphatidylinositol expression and/or phosphatidylinositol cell signaling in a cell include cells which abnormally proliferate, including cancer cells. This includes the abnormal growth and/or proliferation of cancer cells including benign and malignant cells of neoplastic diseases. Inhibition of abnormal cell growth can occur by a variety of mechanisms including, but not limited to, cell death, apoptosis, inhibition of cell division, transcription, translation, transduction, etc. In one embodiment, inhibition of cell growth is facilitated by modulation of the phosphatidylinositol signal transduction pathway by an agent containing a chlorotoxin binding domain leading to alterations in membrane trafficking function (e.g., endocytosis and exocytosis), membrane cytoskeletal function (e.g., microvilli formation and phagocytosis) and cell signaling function (e.g., protein kinase activity, GTPase activity and EGFR-dependent membrane ruffling).

The methods of the invention also include selectively modulating any of the enzymes involved in the phosphatidylinositol signal pathway in the clinical setting in order to ameliorate disease or disorders mediated by activity of any of these enzymes. Thus, treatment of diseases or disorders characterized by excessive or inappropriate activity of these enzymes can be treated through use of chlorotoxin or derivatives thereof according to the invention. Enzymes involved in the phosphatidylinositol signal pathway whose activity can be modulated include, but is not limited to, phosphoinositide 3-kinase, phosphoinositide 4-kinase, phosphoinositide-3-P-4-kinase, phosphoinositide-4-P-5-kinase, 3-phosphatase and 5-phosphatase.

The methods described herein benefit from the use of chlorotoxin or derivatives thereof that selectively inhibit, and preferably specifically inhibit, the activity of any of the enzymes involved in the phosphatidylinositol signal pathway in cells, including cells in vitro, in vivo or ex vivo. Cells useful in the methods include those that express endogenous phosphoinositols which bind chlorotoxin or derivatives thereof, wherein endogenous indicates that the cells express these enzymes absent recombinant introduction into the cells of one or more polynucleotides encoding enzymes which produce these molecules. In practicing the methods of the invention, the cells can be in vivo (i.e., in a living subject, e.g., an animal or human), wherein chlorotoxin or a derivative thereof can be used as a therapeutic to inhibit activity of one or more enzymes involved in the phosphatidylinositol signal pathway in the subject. Alternatively, the cells can be isolated as discrete cells or in a tissue, for ex vivo or in vitro methods.

As discussed above, the methods of the invention can be utilized in vivo, ordinarily in mammals, such as humans, sheep, horses, cattle, pigs, dogs, cats, rats and mice or in vitro. The invention is particularly useful in the treatment of human subjects.

As used herein, the terms “abnormal cell proliferation” and “disease characterized by cell proliferation” unless otherwise indicated, refer to cell proliferation that is independent of normal regulatory mechanisms (e.g., loss of contact inhibition). This includes the abnormal proliferation and/or growth of cells in both benign and malignant cells of neoplastic diseases. Inhibition of abnormal cell proliferation can occur by a variety of mechanisms including, but not limited to, cell death, apoptosis, arrest of mitosis, inhibition of cell division, transcription, translation, transduction, etc. In one embodiment, the inhibition of abnormal cell proliferation occurs by modulation of the phosphatidylinositol signal transduction pathway following binding of chlorotoxin or a derivative thereof to one or more species of phosphatidylinositol leading to arrest of cell division at the G1/S phase of the cell cycle.

As used herein, an “effective amount” of chlorotoxin is an amount which specifically binds to a phosphatidylinositol and exerts an effect on cells exhibiting abnormal growth, such as cancer cells. Examples of phosphatidylinositols to which chlorotoxin or derivatives thereof specifically bind to, include, but are not limited to mono, bis and tris phosphates. Representative monophosphates include, but are not limited to phosphatidylinositol 3-phosphate, phosphatidylinositol 4-phosphate and phosphatidylinositol 5-phosphate. Representative bisphosphates include, but are not limited to phosphatidylinositol 3,4-bisphosphate, phosphatidylinositol 3,5-bisphosphate and phosphatidylinositol 4,5-bisphosphate. Representative trisphosphates include, but are not limited to phosphatidylinositol 3,4,5-trisphosphate.

As used herein, compositions comprising, treatment with or administration of “chlorotoxin” includes to the same degree treatment with chlorotoxin analogues, derivatives, fragments, variants, related peptides and mimetics disclosed herein.

In one embodiment of the methods of the invention, the abnormal cell proliferation is cancer. As used herein, the term “cancer” unless otherwise indicated, refers to diseases that are characterized by uncontrolled, abnormal cell proliferation and/or growth. Types of cancer where the compositions are useful include, but are not limited to, prostate cancer, breast cancer, lung cancer, non-small cell lung carcinoma, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancers, spinal axis tumors, myeloid tumors, glioma, meningioma, pituitary adenoma, or a combination of one or more of the foregoing cancers.

In another embodiment of the compositions and methods of the invention, the abnormal cell proliferation is a benign proliferative disease, including, but not limited to, benign prostatic hyperplasia, hypertrophy, age-related macular degeneration, diabetic retinopathy or restinosis.

As used herein, the term “chlorotoxin” unless otherwise described, refers to the full-length, thirty-six amino acid polypeptide naturally derived from Leiurus quinquestriatus scorpion venom (DeBin et al. (1993) Am. J. Physiol. 264, C361-369) which comprises the amino acid sequence of native chlorotoxin as set forth in SEQ ID NO: 1. The term “chlorotoxin” includes polypeptides comprising SEQ ID NO: 1 which have been synthetically or recombinantly produced, such as those disclosed in U.S. Pat. No. 6,319,891, which is herein incorporated by reference in its entirety.

As used herein, the term “chlorotoxin binding domain” or “binding domain of chlorotoxin” refers to a portion of a molecule which interacts with a phosphatidylinositol to block the binding of chlorotoxin to the phosphatidylinositol. In some embodiments the chlorotoxin binding domain is a small molecule or peptide mimetic, while in other embodiments it is a peptide. Examples of peptides include, but are not limited to, the beta (SEQ ID NO: 8) binding domain of chlorotoxin.

As used herein, the term “agent comprising a chlorotoxin binding domain” refers to any molecule which is capable of specifically blocking the binding of chlorotoxin to one or more species of phosphatidylinositols. Examples include, but are not limited to small molecules, polypeptides, antibodies, peptide mimetics, etc.

As used herein, the term “chlorotoxin derivative” refers to derivatives, analogs, variants, polypeptide fragments and mimetics of chlorotoxin and related peptides which retain the same activity as chlorotoxin, such as binding specifically binding to a cancer cell when compared to a normal cell, can also be used for practicing the methods of the invention. Examples of derivatives include, but are not limited to, peptide variants of chlorotoxin, peptide fragments of chlorotoxin, for example, fragments comprising or consisting of contiguous 10-mer peptides of SEQ ID NO: 1, 2, 3, 4, 5, 6 or 7 or comprising about residues 10-18 or 21-30 of SEQ ID NO: 1, core binding sequences, and peptide mimetics.

Chlorotoxin and peptide derivatives thereof can be prepared using standard solid phase (or solution phase) peptide synthesis methods, as is known in the art. In addition, the nucleic acids encoding these peptides may be synthesized using commercially available oligonucleotide synthesis instrumentation and produced recombinantly using standard recombinant production systems. The production using solid phase peptide synthesis is necessitated if non-gene-encoded amino acids are to be included. The term “chlorotoxin derivative” as used herein is synonymous with “variant” also includes modifications to the chlorotoxin sequence by one or more deletions of up to ten (e.g., one to seven or one to five amino acids; insertions of a total of up to ten (e.g., one to five) amino acids internally within the amino acid sequence of chlorotoxin; or of up to a total of one-hundred amino acids at either terminus of the chlorotoxin sequence; or conservative substitutions of a total of up to fifteen (e.g., one to five) amino acids.

Derivatives of chlorotoxin include polypeptides comprising a conservative or non-conservative substitution of at least one amino acid residue when the derivative sequence and the chlorotoxin sequence are maximally aligned. The substitution may be one which enhances at least one property or function of chlorotoxin, inhibits at least one property or function of chlorotoxin, or is neutral to at least one property or function of chlorotoxin. As used herein, a “property or function” of chlorotoxin includes, but is not limited to, binding to phosphatidylinositols leading to modulation of the phosphatidylinositol signal transduction pathway (Czech et al. (2000) Cell 100, 603-606). Modulation of this pathway is also associated with alterations in membrane trafficking function, membrane-cytoskeletal function or cell signaling function, all of which can be attributed to binding of chlorotoxin to one or more phosphatidylinositols. Additional properties or functions can also be selected from the group consisting of the ability to arrest abnormal cell growth, cause paralysis of a subject, specific binding to a benign or malignant cancer cell when compared to a non-cancer cell (i.e., normal), and killing of a benign or malignant cancer cell. In terms of the present disclosure, the cancer cell may be in vivo, ex vivo, in vitro, a primary isolate from a subject, a cultured cell or a cell line. Derivatives of chlorotoxin further include polypeptides comprising the amino acid sequence KGRGKSY (SEQ ID NO: 8), corresponding to amino acid residues 23-29 of SEQ ID NO: 1 (also known as the beta domain), and/or TDHQMAR (SEQ ID NO: 9) residing at amino acid residues 8-14 of SEQ ID NO: 1 (also known as the alpha domain).

Peptide variants of chlorotoxin include, but are not limited to, deletion or conservative amino acid substitution variants of SEQ ID NO: 1. As used herein, a conservative variant refers to alterations in the amino acid sequence that do not adversely substantially affect the biological functions of the peptide. A substitution, insertion or deletion is said to adversely affect the peptide when the altered sequence substantially prevents or disrupts a biological function associated with the peptide (e.g., binding to a cancer cell). For example, the overall charge, structure or hydrophobic/hydrophilic properties of the peptide can be altered without adversely affecting a biological activity. Accordingly, the amino acid sequence can be altered, for example to render the peptide more hydrophobic or hydrophilic, without adversely affecting the biological activities of the peptide.

The methods of the invention include corresponding polypeptide toxins of other scorpion species that display similar or related activity to chlorotoxin for the diagnosis and treatment of diseases associated with abnormal cell proliferation as described herein, including cancer. For purposes of the specification, “similar or related activity to chlorotoxin” is defined as binding to cells displaying abnormal cell growth, including benign cells exhibiting abnormal growth and malignant cancer cells. Examples of such polypeptide toxins include, but are not limited to, toxins which contain one or more of the binding domains of chlorotoxin set forth in SEQ ID NO: 8 or SEQ ID NO: 13, and any of the consensus sequences set forth in Table 1.

As used herein, the term “related scorpion toxin” refers to any of the toxins or related peptides displaying amino acid and/or nucleotide sequence identity to chlorotoxin. Examples of related scorpion toxins include, but are not limited to, CT neurotoxin from Mesobuthus martensii (GenBank Accession AAD47373), Neurotoxin BmK 41-2 from Buthus martensii karsch (GenBank Accession A59356), Neurotoxin Bm12-b from Buthus martensii (GenBank Accession AAK16444), Probable Toxin LQH 8/6 from Leiurus quinquestriatus hebraeu (GenBank Accession P55966), Small toxin from Mesobuthus tamulus sindicus (GenBank Accession P15229), the sequences of which are all herein incorporated by reference in their entirety.

Homology or sequence identity at the nucleotide or amino acid sequence level is determined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89,10915-10919, fully incorporated by reference), recommended for query sequences over eighty-five nucleotides or amino acids in length.

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The methods of the present invention encompass use of allelic variants, conservative substitution variants, and the members of the scorpion toxin peptide family, having an amino acid sequence of at least about seventy-five percent, at least about eighty-five percent, at least about ninety percent sequence, at least about ninety-five percent, or at least about ninety-nine percent sequence identity with the entire chlorotoxin sequence set forth in SEQ ID NO: 1. Identity or homology with respect to such sequences is defined herein as the percentage of amino acid residues in the candidate sequence that are identical with the known peptides, after alignment the sequences.

Fusion proteins, or N-terminal, C-terminal or internal extensions, deletions, or insertions into the peptide sequence shall not be construed as affecting homology. Examples of such extensions include, but are not limited to, the following sequences:

(SEQ ID NO: 2)

HHHHHHMCMPCFTTDHQMARKCDDCCGGKGRGKCYGPQCLCR,

(SEQ ID NO: 3)

YMCMPCFTTDHQMARKCDDCCGGKGRGKCYGPQCLCR,

(SEQ ID NO: 4)

YSYMCMPCFTTDHQMARKCDDCCGGKGRGKCYGPQCLCR.

The chlorotoxin peptide variants include peptides having a fragment of the amino acid sequence set forth in SEQ ID NO: 1, having at least about 7, 8, 9, 10, 15, 20, 25, 30, or 35 contiguous amino acid residues. The peptide variants further include those fragments associated with the activity of chlorotoxin. Such fragments, also referred to as polypeptides, may contain functional regions of the chlorotoxin peptide identified as regions of the amino acid sequence which correspond to known peptide domains, as well as regions of pronounced hydrophilicity. Variants may also include peptide with at least two core sequences linked to one another, in any order, with intervening amino acids removed or replaced by a linker sequence. The regions are all easily identifiable by using commonly available protein sequence analysis software such as MacVector (Oxford Molecular).

Contemplated peptide variants further include those containing predetermined mutations by, e.g., homologous recombination, site-directed or PCR mutagenesis, and the alleles or other naturally occurring variants of the family of peptides; and derivatives wherein the peptide has been covalently modified by substitution, chemical, enzymatic or other appropriate means with a moiety other than a naturally occurring amino acid (for example a detectable moiety such as an enzyme or radioisotope). Examples of chlorotoxin variant peptides include, but are not limited to the following sequences:

(SEQ ID NO: 5)

MCMPCFTTDHQMARKCDDCCGGKGRGKCFGPQCLCR,

(SEQ ID NO: 6)

RCKPCFTTDPQMSKKCADCCGGKGKGKCYGPQCLC,

(SEQ ID NO: 7)

RCSPCFTTDQQMTKKCYDCCGGKGKGKCYGPQCICAPY.

In practicing the methods of the invention, chlorotoxin and/or derivatives thereof may be used alone or in combination with other inactive ingredients. As discussed above, the present invention includes compositions and methods where a drug or cytotoxic agent is linked to a chlorotoxin derivative. The methods of the invention therefore include administration of a chlorotoxin derivative linked to a cytotoxic agent for the treatment of a disease associated with abnormal cell growth, including cancer. Examples of cytotoxic agents include, but are not limited to, gelonin, ricin, saponin, pseudonomas exotoxin, pokeweed antiviral protein, diphtheria toxin, complement proteins, or any other agent known in the art which is capable of killing a cell upon contact with that cell.

Chlorotoxin Peptide Mimetics

In another class of chlorotoxin derivatives, the present methods include use of mimetics, including peptide mimetics, that mimic the three-dimensional structure of chlorotoxin. Such mimetics may have significant advantages over naturally occurring peptides including, for example, more economical production, greater chemical stability, enhanced pharmacological properties (half-life, absorption, potency, efficacy, etc.), altered specificity (e.g., a broad-spectrum of biological activities), reduced antigenicity and others.

In one form, mimetics are peptide-containing molecules that mimic elements of chlorotoxin peptide secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly produced in the pharmaceutical industry as non-peptide drugs with properties analogous to those of the template peptide. These types of non-peptide compounds are also referred to as peptide mimetics or peptidomimetics (Fauchere (1986) Adv. Drug Res. 15, 29-69; Veber & Freidinger (1985) Trends Neurosci. 8,392-396; Evans et al. (1987) J. Med. Chem. 30, 1229-1239 which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage by methods known in the art. Labeling of peptide mimetics usually involves covalent attachment of one or more labels, directly or through a spacer (e.g., an amide group), to non-interfering positions on the peptide mimetic that are predicted by quantitative structure-activity data and molecular modeling. Such non-interfering positions generally are positions that do not form direct contacts with the macromolecules to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of a peptide to, for instance, a tumor cell. Approaches that can be used include the yeast two hybrid method (see Chien et al. (1991) Proc. Natl. Acad. Sci. USA 88, 9578-9582) and using the phage display method. The two hybrid method detects protein-protein interactions in yeast (Fields et al. ( 1989) Nature 340, 245-246). The phage display method detects the interaction between an immobilized protein and a protein that is expressed on the surface of phages such as lambda and M13 (Amberg et al. (1993) Strategies 6, 2-4; Hogrefe et al. (1993) Gene 128, 119-126). These methods allow positive and negative selection for peptide-protein interactions and the identification of the sequences that determine these interactions.

Pharmaceutical Compositions

The methods of the present invention encompass the use of pharmaceutical compositions that can be administered via parenteral, subcutaneous, intravenous, intramuscular, intraperitoneal, intrathecal, intracranial or transdermal or buccal routes. For example, an agent may be administered locally to a tumor via microinfusion. Alternatively, or concurrently, administration may be by the oral route. The dosage administered will be dependent upon the age, health, and weight of the recipient, kind of concurrent treatment, if any, frequency of treatment, and the nature of the effect desired.

While individual needs vary, determination of optimal ranges of effective amounts of each component is within the skill of the art. Dosages of chlorotoxin and/or derivatives thereof of the present invention typically comprise about 1.0 ng/kg body weight to about 0.13 mg/kg body weight. In one embodiment, dosages of chlorotoxin and/or derivatives thereof comprise about 1.0 ng/kg body weight to about 0.1 mg/kg body weight. In a preferred embodiment, dosages for systemic administration comprise about 0.01 μg/kg body weight to about 0.1 mg/kg body weight. In another embodiment, the dosage of chlorotoxin and/or derivatives thereof comprises less than about 0.1 mg/kg body weight. More preferred dosages for systemic administration comprise about 0.1 μg/kg body weight to about 0.05 mg/kg body weight. In another preferred embodiment, the dosage of chlorotoxin and/or derivatives thereof comprises less than about 0.05 mg/kg body weight. The most preferred dosages for systemic administration comprise between about 1.0 μg/kg body weight to about 0.01 mg/kg body weight. In other embodiments, the amount of chlorotoxin and/or derivatives thereof administered is an amount effective to bring the concentration of chlorotoxin and/or derivatives thereof in the serum to a concentration of about 20.0, 10.0, 5.0, 2.50, 1.25, 0.625, 0.3125, 0.156, 0.078, 0.039, 0.020, 0.010, 0.005, 0.003, 0.0015, 0.0008, 0.0003 or 0.0001 nM. The preferred dosages for direct administration to a site via microinfusion comprise 1 ng/kg body weight to 1 mg/kg body weight.

In addition to agents comprising a chlorotoxin binding domain, the methods of the present invention encompass the use of suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil or synthetic fatty acid esters, for example, ethyl oleate or triglycerides. Aqueous injection suspensions may contain substances which increase the viscosity of the suspension including, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration may be formulated for enteral, parenteral or topical administration. Indeed, all three types of formulations may be used simultaneously to achieve systemic administration of the active ingredient.

As mentioned above for some methods of the invention, topical administration may be used. Any common topical formulation such as a solution, suspension, gel, ointment or salve and the like may be employed. Preparation of such topical formulations are described in the art of pharmaceutical formulations as exemplified, for example, by Gennaro et al. (1995) Remington's Pharmaceutical Sciences, Mack Publishing. For topical application, the compositions could also be administered as a powder or spray, particularly in aerosol form. In a some embodiments, the compositions of this invention may be administered by inhalation. For inhalation therapy the active ingredients may be in a solution useful for administration by metered dose inhalers or in a form suitable for a dry powder inhaler. In another embodiment, the compositions are suitable for administration by bronchial lavage.

Suitable formulations for oral administration include hard or soft gelatin capsules, pills, tablets, including coated tablets, elixirs, suspensions, syrups or inhalations and controlled release forms thereof.

The methods of the invention also include the use of isotopically-labeled agents with a chlorotoxin binding domain that have one or more atoms replaced by an atom having an atomic mass or mass number different from the atomic mass or mass number usually found in nature, or one or more of such atoms attached to the chlorotoxin derivatives. Examples of isotopes that can be incorporated into compounds of the invention include, but are not limited to, isotopes of hydrogen, carbon, phosphorous, iodine, rhenium, indium, yttrium, technetium and lutetium (i.e., including, but not limited to, ³H, ¹⁴C, ³¹P, ³²P, ³⁵S, ¹³¹I, ¹²⁵I, ¹²³I, ¹⁸⁷Re, ⁶⁴Cu, ¹¹¹In, ⁹⁰Y, ^{99m}Tc, ¹⁷⁷Lu), others isotopes of these elements, and other isotopes known in the art. Agents of the present invention, prodrugs thereof, and pharmaceutically acceptable salts of said agents or of said prodrugs which contain the aforementioned isotopes and/or other isotopes of other atoms are within the scope of this invention. Tritium and carbon-14 isotopes are particularly preferred for their ease of preparation and detectability. Further, substitution with heavier isotopes such as deuterium can afford certain therapeutic advantages resulting from greater metabolic stability, for example increased in vivo half-life or reduced dosage requirements and, hence, may be preferred in some circumstances.

Fusion Proteins

The methods of the invention also include the use of compositions where a cytotoxic agent is linked to an agent comprising a chlorotoxin binding domain. Examples of cytotoxic agents include, but are not limited to, gelonin, ricin, saponin, pseudonomas exotoxin, pokeweed antiviral protein, diphtheria toxin, complement proteins, or any other agent known in the art which is capable of killing a cell upon contact with that cell.

The methods of the invention also include the use of fusion polypeptides and salts thereof, comprising at least one second polypeptide. In some embodiments, the second polypeptide includes a cancer cell-binding domain which specifically binds to an epitope expressed only on cells exhibiting abnormal growth (i.e. cancer cells). The term “cancer cell-binding domain” refers to an amino acid sequence capable of binding or otherwise specifically associating with a cell displaying abnormal growth (e.g., benign and malignant cancer cells). In some embodiments the cancer cell binding-domain is an antibody while in other embodiments it is a ligand which specifically binds to a receptor expressed only on cancer cells. Examples of antibodies include, but are not limited to, antibodies which specifically bind to B-cells or T-cells. Examples of receptor ligands include, but are not limited to, cytokines and growth factors including epidermal growth factor.

The second polypeptide can also include a stabilization domain which increases the in vitro and in vivo half-life of the fusion polypeptide. As used herein, the term “stabilization domain” refers to an amino acid sequence capable of extending the in vitro and in vivo half-life of chlorotoxin or a chlorotoxin derivative when compared to chlorotoxin alone. The stabilization domain can comprise human proteins (e.g., full length or truncated, soluble proteins from extracellular fragments, etc) such as human serum albumin, transferrin or other proteins which stabilize the in vivo or in vitro half-life of chlorotoxin or a chlorotoxin derivative. These additional functional domains may themselves serve as linker peptides, for example, for joining a cancer cell-binding domain to chlorotoxin or a chlorotoxin derivative. Alternatively, they may be located elsewhere in the fusion molecule (e.g., at the amino or carboxy terminus thereof). In alternative embodiments, the stabilization domain is a chemical moiety (e.g., PEG (polyethylene glycol) or a dextran).

The term “fused” or “fusion polypeptide” as used herein refers to polypeptides in which: (i) a given functional domain (i.e. a cancer cell-binding domain) is bound at its carboxy terminus by a covalent bond either to the amino terminus of another functional domain (i.e., an human serum albumin component) or to a linker peptide which itself is bound by a covalent bond to the amino terminus of chlorotoxin or a chlorotoxin derivative; or (ii) a given functional domain (i.e. a cancer cell-binding domain) is bound at its amino terminus by a covalent bond either to the carboxy terminus of another functional domain (i.e., an human serum albumin component) or to a linker peptide which itself is bound by a covalent bond to the carboxy terminus of chlorotoxin or a chlorotoxin derivative.

Similarly, “fused” when used in connection with the nucleic acid intermediates of the invention means that the 3′- [or 5′-] terminus of a nucleotide sequence encoding a first functional domain is bound to the respective 3′- [or 5′-] terminus of a nucleotide sequence encoding a second functional domain, either by a covalent bond or indirectly via a nucleotide linker which itself is covalently bound preferably at its termini to the first functional domain-encoding polynucleotide and optionally, a second functional domain-encoding nucleic acid.

Examples of fusion polypeptides of the invention may be represented by, but are not limited by, the following formulas:
R1-L-R2 (i)
R2-L-R1 (ii)
R1-L-R2-L-R1 (iii)
R1-L-R1-L-R2 (iv)
R2-L-R1-L-R1 (iv)
wherein R1 is the amino acid sequence of a cancer cell-binding domain, R2 is the amino acid sequence of a stabilizing domain (e.g., human serum albumin), each L is chlorotoxin or a chlorotoxin derivative which is bound by a covalent bond to a terminus of R1 and/or R2, whereby the above molecule fragments are read directionally (i.e., with the left side corresponding to the amino terminus and the right side to the carboxy terminus of the molecule).
Methods to Identify Binding Partners

Another embodiment of the present invention provides methods for use in isolating and identifying binding partners of the phosphatidylinositols which binds an agent comprising a chlorotoxin binding domain. In one embodiment, the agent is chlorotoxin. In general, a protein of the invention is mixed with a potential binding partner or an extract or fraction of a cell under conditions that allow the association of potential binding partners with the phosphatidylinositols in the presence and absence of an agent containing a chlorotoxin binding domain. After mixing, peptides, polypeptides, proteins or other molecules that have become associated with a protein of the invention are separated from the mixture. The binding partner that bound to the protein of the invention can then be removed and further analyzed. To identify and isolate a binding partner, purified species of phosphatidylinositols can also be used, for instance a phosphatidylinositol-monophosphate, phosphatidylinositol-bisphosphate or a phosphatidylinositol-trisphosphate. Such species of phosphatidylinositols are commercially available, either in free form or linked to acrylic beads, and are well suited for use in methods to identify binding partners.

As used herein, a cellular extract refers to a preparation or fraction that is made from a lysed or disrupted cell. The preferred source of cellular extracts will be cells derived from human tumor tissue, including neuroectodermal tumor tissue. Alternatively, cellular extracts may be prepared from available cell lines, particularly cancer cell lines, including those cell lines derived from glioma and meningioma tumors. A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the test agent under conditions in which association of a binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a phospholipid can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture. To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins. Alternatively, binding partners may be identified using a Far-Western assay according to the procedures of Takayama et al. (1997) Methods Mol. Biol. 69, 171-184 or Sauder et al. (1996) J. Gen. Virol. 77, 991-996 or identified through the use of epitope tagged proteins or GST fusion proteins.

Alternatively, the nucleic acid molecules of the invention can be used in a yeast two-hybrid system. The yeast two-hybrid system has been used to identify other protein partner pairs and can readily be adapted to employ the nucleic acid molecules herein described.

Methods to Identify Agents that Modulate Expression

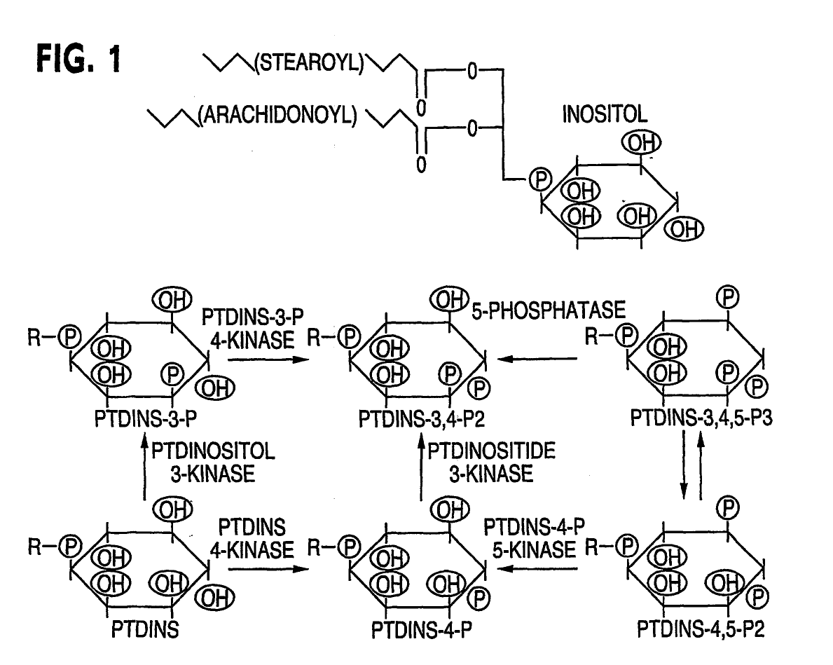

Another embodiment of the present invention provides methods for identifying agents that modulate the expression of the phosphatidylinositols which bind chlorotoxin. Such assays may utilize any available means of monitoring for changes in the expression level of these phosphatidylinositols. As used herein, an agent is said to modulate the expression of a phosphatidylinositol if it is capable of up- or down-regulating expression of the phosphatidylinositol by a cell or any of the enzymes responsible for synthesis of these phosphatidylinositols. Exemplary enzymes include, but are not limited to those enzymes depicted in FIG. 1.

Probes to detect differences in expression levels between cells exposed to the agent and control cells may be prepared. In one assay format, cells or cell lines are first identified which express the phosphoinositols which bind chlorotoxin physiologically. Cell and/or cell lines so identified would be expected to comprise the necessary cellular machinery such that the fidelity of the appropriate surface transduction mechanisms and/or the cytosolic cascades is maintained.

Cells or cell lines are then contacted with test agents under appropriate conditions; for example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37EC. Said conditions may be modulated as deemed necessary by one of skill in the art. Subsequent to contacting the cells with the agent, said cells will be disrupted and the phospholipids of the lysate are fractionated such that a phosphatidylinositol fraction is pooled and contacted with an antibody to be further processed by immunological assay (e.g., ELISA, immunoprecipitation or Western blot). The pool of proteins isolated from the “agent-contacted” sample will be compared with a control sample where only the excipient and/or chlorotoxin or a derivative thereof is contacted with the cells and an increase or decrease in the immunologically generated signal from the agent-contacted sample compared to the control will be used to distinguish the effectiveness of the agent.

Methods to Identify Agents that Modulate Activity

The present invention provides methods for identifying agents that modulate at least one activity associated with the phosphatidylinositols which bind chlorotoxin. Such methods or assays may utilize any means of monitoring or detecting the desired activity.

In one format, the specific activity of a protein of the invention, normalized to a standard unit, between a cell population that has been exposed to the agent to be tested compared to an un-exposed control cell population may be assayed. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the phosphatidylinositols which bind chlorotoxin. To enhance immunogenicity, these phosphatidylinositols can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents may be desirable to provide accessibility to the hapten. Administration of the phosphatidylinositols is conducted generally by injection over a suitable time period and with use of suitable adjuvants, as is generally understood in the art. During the immunization schedule, titers of antibodies are taken to determine adequacy of antibody formation.

Use of monoclonal antibody preparations which bind to particular phosphatidyinositols is also contemplated within the methods of the invention. Immortalized cell lines which secrete the desired monoclonal antibodies may be prepared using standard methods (see e.g., Kohler & Milstein (1992) Biotechnology 24, 524-526) or modifications which effect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies may be recovered from the culture supernatant or from the ascites supernatant. Fragments of the monoclonal antibodies or the polyclonal antisera that contain the immunologically significant portion can be used in the methods of the invention, as well as the intact antibodies. Use of immunologically reactive fragments, such as Fab or Fab′ fragments is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin. Antibody regions that bind specifically to the desired regions of the protein can also be produced in the context of chimeras with multiple species origin, for instance, humanized antibodies. The antibody can therefore be a humanized antibody or, a human antibody, as described in U.S. Pat. No. 5,585,089 or Riechmann et al. (1988) Nature 332, 323-327.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any chlorotoxin binding domain.

The agents of the present invention can be, as examples, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, nucleic acid molecules encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies or fragments thereof that bind to the phosphoinositols that bind chlorotoxin. Antibody agents can be obtained by immunization of suitable mammalian subjects with peptides, containing as antigenic regions, those portions of the protein intended to be targeted by the antibodies.

In yet another class of agents, the present invention includes peptide mimetics that mimic the three-dimensional structure of chlorotoxin or derivatives thereof that bind to phosphoinositols. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others.

In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

In another form, peptide analogs are commonly used in the pharmaceutical industry as non-peptide drugs with properties analogous to those of the template peptide. These types of non-peptide compounds are also referred to as peptide mimetics or peptidomimetics (Fauchere (1986) Adv. Drug Res. 15, 29-69; Veber & Freidinger (1985) Trends Neurosci. 8, 392-396; Evans et al. (1987) J. Med. Chem. 30, 1229-1239 which are incorporated herein by reference) and are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage by methods known in the art.

Labeling of peptide mimetics usually involves covalent attachment of one or more labels, directly or through a spacer (e.g., an amide group), to non-interfering positions on the peptide mimetic that are predicted by quantitative structure-activity data and molecular modeling. Such non-interfering positions generally are positions that do not form direct contacts with the macromolecules to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al. (1991) Proc. Natl. Acad. Sci. USA 88, 9578-9582) and using the phage display method. The two hybrid method detects protein-protein interactions in yeast (Fields et al. (1989) Nature 340, 245-246). The phage display method detects the interaction between an immobilized protein and a protein that is expressed on the surface of phages such as lambda and M13 (Amberg et al. (1993) Strategies 6, 2-4; Hogrefe et al. (1993) Gene 128, 119-126). These methods allow positive and negative selection for protein-protein interactions and the identification of the sequences that determine these interactions.

Without further description, it is believed that one of ordinary skill in the art can, using the preceding description and the following illustrative examples, make and utilize the compounds of the present invention and practice the claimed methods. The following working examples describe embodiments of the present invention, and are not to be construed as limiting in any way the remainder of the disclosure.

### EXAMPLE 1

Binding of Chlorotoxin to Phosphatidylinositols

Sedimented suspensions of PIP beads™ were obtained from Molecular Probes. The beads are supplied as 10 nanomoles of bound phosphoinositide per milliliter of sedimented suspension. PIP beads were pelleted by centrifugation at low speed in a Beckman Coulter Microfuge. Pelleted beads were washed in wash-binding buffer (0.5% NP-40 in phosphate-buffered saline, 1 ml, 3×5 minutes) and centrifuged at low speed following each wash. After washing, pelleted beads were resuspended in an equal volume of Wash/Binding buffer. 100 μl of bead suspension was used for each binding reaction. Biotinylated chlorotoxin was diluted in wash-binding buffer, added to each phosphatidylinositol bead suspension at a final concentration of 100 μM and allowed to incubate for two hours at room temperature. Following incubation with chlorotoxin, the bead suspensions were washed in Wash/Binding buffer (1 ml, 3×5 minutes) and centrifuged at low speed after each wash. Streptavidin horseradish peroxidase (Vector Labs) was diluted 1:1000 in wash-binding buffer, 100 μl of this solution was added to each of the phosphatidylinositol bead suspensions, and allowed to incubate one at room temperature. Following incubation with streptavidin, bead suspensions were washed thoroughly in Wash/Binding buffer (1 ml, 5×10 minutes) and centrifuged at low speed after each wash. Immediately prior to use, a 1:1 mixture of TMB substrate (Pierce) was prepared, 100 μl of this solution was added to each reaction, and allowed to incubate at room temperature for up to thirty minutes. After incubation with substrate, bead suspensions were pelleted by centrifugation, supernatants were transferred to a 96-well plate and absorbance (λ=595 nm) was obtained using BioRad microplate reader and microplate manager software.

### EXAMPLE 2

Binding of Chlorotoxin Derivatives to Phosphatidylinositols

Chlorotoxin is a 36-amino acid peptide with 8 cysteines, depicted below in bold type with the sequences of beta-domain peptide (SEQ ID NO: 8) and alpha-domain peptide (SEQ ID NO: 9) underlined below:

(SEQ ID NO: 1)

MCMPCFTTDHQMARKCDDCCGGKGRCKCYGPQCLCR

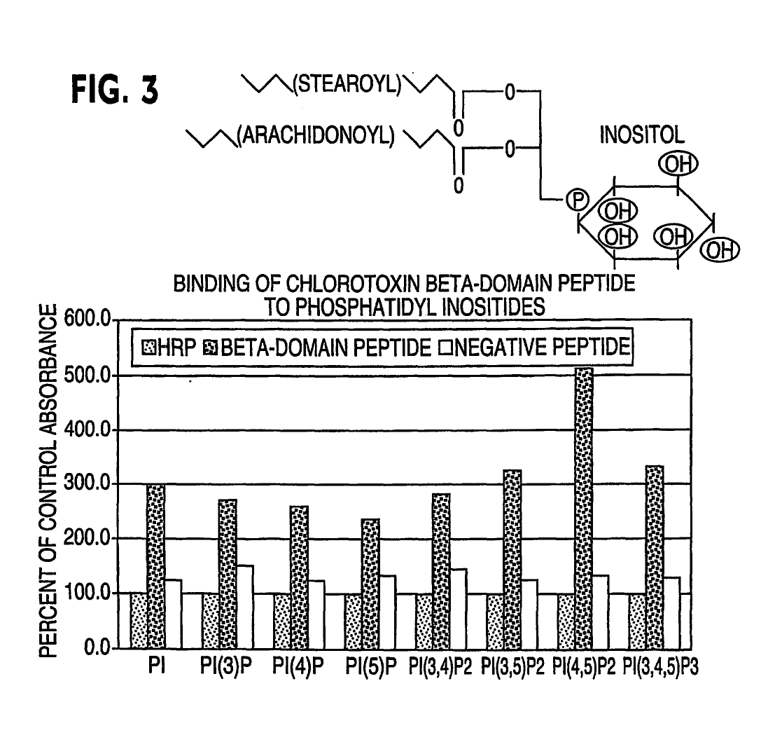

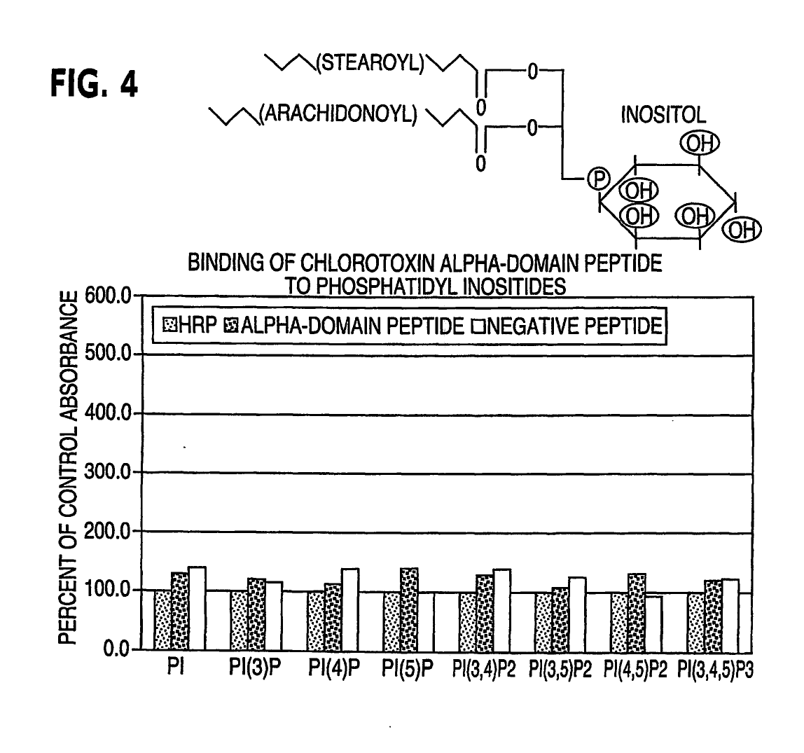

In order to confirm the identify of the minimal binding sequences within the alpha and beta peptides, each of the two peptides was incubated with different species of phosphatidylinositols as set forth in Example 1. Results demonstrated that the beta-domain peptide binds to multiple species of phosphatidylinositols (FIG. 3) while the alpha-domain peptide displayed no binding activity (FIG. 4). The beta-domain peptide bound to all of the same species of phosphatidylinositols as did chlorotoxin including mono, bis (e.g., phosphatidylinositol-4,5-bisphosphate) and tris phosphates.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

We claim:

1. A method of inhibiting a phosphatidylinositol signaling cascade in a cell comprising contacting a cell expressing a phosphatidylinositol phospholipid with an effective amount of an agent comprising a chlorotoxin binding domain, wherein the chlorotoxin binding domain binds to the phosphatidylinositol phospholipid.

2. The method of claim 1 further comprising contacting the agent with a phosphatidylinositol phospholipid in vitro to determine the effective amount of the agent.

3. A method of inhibiting a phosphatidylinositol signaling cascade in a cell comprising contacting a cell expressing a phosphatidylinositol phospholipid with an agent comprising a chlorotoxin binding domain, wherein the agent is not full length chlorotoxin.

4. The method of claim 1 wherein the agent inhibits one or more plasma membrane functions that require the phosphatidylinositol phospholipid.

5. The method of claim 4 wherein the plasma membrane function is selected from the group consisting of a membrane trafficking function, a membrane-cytoskeletal function and a cell signaling function.

6. The method of claim 5 wherein the membrane trafficking function is selected from the group consisting of endocytosis and exocytosis.

7. The method of claim 4 wherein the membrane-cytoskeletal function is selected from the group consisting of microvilli formation and phagocytosis.

8. The method of claim 4 wherein the cell signaling function is selected from the group consisting of protein kinase activity, GTPase activity and EGFR-dependent membrane ruffling.

9. The method of claim 1 wherein the inhibition of the phosphatidylinositol signaling cascade is effective for the treatment of cancer.

10. A method of inhibiting the activity of a cancer cell expressing a phosphatidylinositol phospholipid on the cell surface comprising contacting the cell with an agent comprising a chlorotoxin binding domain.

11. The method of claim 10 wherein the activity is cancer cell division.

12. The method of claim 10 wherein the cancer cell division is arrested at the G1/S phase of the cell cycle.

13. The method of claim 10 wherein the activity is the phosphotidylinositol cell signaling pathway.

14. The method of claim 10 wherein the inhibition of cancer cell activity is effective for the treatment of cancer.

15. A method of treating cancer in a patient in need of such treatment comprising administering an effective amount of a composition comprising an agent containing a chlorotoxin binding domain that binds to a cancer cell expressing a phosphatidylinositol phospholipid.

16. The method of claim 15 further comprising contacting the agent with a phosphatidylinositol phospholipid in vitro to determine the effective amount of the agent.

17. A method of treating cancer in a patient in need of such treatment comprising administering an effective amount of a composition comprising an agent containing a chlorotoxin binding domain that binds to a cancer cell expressing a phosphatidylinositol phospholipid, wherein the agent is not full length chlorotoxin.

18. The method of claim 1, 10, 15 or 17 wherein the phosphotidylinositol phospholipid is a monophosphate.

19. The method of claim 18 wherein the phosphotidylinositol monophosphate is phosphatidyinositol 3-phosphate or phosphatidyinositol 4-phosphate.

20. The method of claim 1, 10, 15 or 17 wherein the phosphotidylinositol phospholipid is a bisphosphate.

21. The method of claim 20 wherein the bisphosphate is phosphatidylinositol 3,4-bisphosphate or phosphatidylinositol 4,5-bisphosphate.

22. The method of claim 1, 10, 15 or 17 wherein the phosphotidylinositol phospholipid is a trisphosphate.

23. The method of claim 22 wherein the trisphosphate is phosphatidylinositol 3,4,5-trisphosphate.

24. The method of claim 1, 10, 15 or 17 wherein the agent is a polypeptide.

25. The method of claim 1, 10, 15 or 17 wherein the agent comprising a chlorotoxin binding domain is chlorotoxin.

26. The method of claim 1, 10, 15 or 17 wherein the agent is a chlorotoxin peptide mimetic.

27. The method of claim 24 wherein the polypeptide comprises at least two chlorotoxin binding domain capable of binding to a phosphotidylinositol phospholipid.

28. The method of claim 24 wherein the polypeptide comprising a chlorotoxin binding domain comprises the amino acid sequence KGRGKSY (SEQ ID NO: 8).

29. The method of claim 1, 10, 15 or 17 wherein the agent is suitable for use in humans.

30. The method of claim 1, 10, 15 or 17 wherein the cancer is selected from a cancer selected from the group consisting of lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), neuroectodermal cancer, spinal axis tumors, glioma, meningioma and pituitary adenoma.

31. A method of identifying an agent which binds to phosphatidylinositol phospholipid comprising:
(a) contacting the phosphatidylinositol phospholipid with the agent in the presence of a polypeptide containing a chlorotoxin binding domain, and
(b) detecting the binding of the agent to the phosphatidylinositol phospholipid.

32. The method of claim 31 further comprising measuring the level of binding of the agent to the phosphatidylinositol phospholipid.

33. The method of claim 31 wherein the method further comprises comparison to a control.

34. The method of claim 33 wherein the control is a negative control that does not contain a phosphatidylinositol phospholipid.

35. The method of claim 33 wherein the control is a positive control which comprises a phosphatidylinositol phospholipid contacted with chlorotoxin.

36. The method of claim 35 wherein the chlorotoxin is labeled.

37. The method of claim 31 wherein the phosphatidylinositol phospholipid is expressed on the surface of cells.

38. The method of claim 37 wherein the cells are exposed to the agent in vitro.

39. The method of claim 37 wherein the cells are eukaryotic or prokaryotic cells.

40. The method of claim 37 further comprising measuring the level of the phosphatidylinositol phospholipid on the surface of the cells.

41. The method of claim 37 further comprising measuring differentiation or proliferation of the cells.

42. The method of claim 37 wherein the cells are cancer cells.

43. The method of claim 37 wherein the cells are disrupted prior to contact with the agent.

44. The method of claim 31 wherein the agent is selected from the group consisting of chemical compounds, oligonucleotides, peptides and antibodies.

45. The method of claim 31 wherein the agent is labeled.

46. The method of claim 45 further comprising measuring binding of the labeled agent to the phosphatidylinositol phospholipid.

47. The method of claim 31 wherein the polypeptide containing a chlorotoxin binding domain is labeled.

48. The method of claim 31 wherein the polypeptide containing a chlorotoxin binding domain is chlorotoxin.

49. The method of claim 31 wherein the polypeptide containing a chlorotoxin binding domain comprises the amino acid sequence KGRGKSY (SEQ ID NO: 8).

50. The method of claim 31 wherein the phosphatidylinositol phospholipid is linked to a bead.

51. The method of claim 31 wherein the wherein the phosphotidylinositol phospholipid is a monophosphate.

52. The method of claim 51 wherein the phosphotidylinositol monophosphate is is phosphatidyinositol 3-phosphate or phosphatidyinositol 4-phosphate.

53. The method of claim 31 wherein the phosphotidylinositol phospholipid is a bisphosphate.

54. The method of claim 53 wherein the bisphosphate is phosphatidylinositol 3,4-bisphosphate or phosphatidylinositol 4,5-bisphosphate.

55. The method of claim 31 wherein the phosphotidylinositol phospholipid is a trisphosphate.

56. The method of claim 55 wherein the trisphosphate is phosphatidylinositol 3,4,5-trisphosphate.


SEQUENCE LISTING (9 sequences)

SEQ ID NO: 1 - protein, 36 aa, Leiurus quinquestriatus
MCMPCFTTDHQMARKCDDCCGGKGRGKCYGPQCLCR

SEQ ID NO: 2 - protein, 42 aa, Artificial Sequence
  Peptide
HHHHHHMCMPCFTTDHQMARKCDDCCGGKGRGKCTGPQCLCR

SEQ ID NO: 3 - protein, 37 aa, Artificial Sequence
  Peptide
YMCMPCFTTDHQMARKCDDCCGGKGRGKCYGPQCLCR

SEQ ID NO: 4 - protein, 39 aa, Artificial Sequence
  Peptide
YSYMCMPCFTTDHQMARKCDDCCGGKGRGKCYGPQCLCR

SEQ ID NO: 5 - protein, 36 aa, Artificial Sequence
  Peptide
MCMPCFTTDHQMARKCDDCCGGKGRGKCFGPQCLCR

SEQ ID NO: 6 - protein, 35 aa, Artificial Sequence
  Peptide
RCKPCFTTDPQMSKKCADCCGGKGKGKCYGPQCLC

SEQ ID NO: 7 - protein, 38 aa, Artificial Sequence
  Peptide
RCSPCFTTDQQMTKKCYDCCGGKGKGKCYGPQCICAPY

SEQ ID NO: 8 - protein, 10 aa, Artificial Sequence
  Peptide
KGRGKCYGPQ

SEQ ID NO: 9 - protein, 9 aa, Artificial Sequence
  Peptide
TDHQMARKC